(19) United States
(12) Patent Application Publication
(10) Pub. No.: US 2021/0299062 A1
(43) Pub. Date: Sep. 30, 2021

(54) CANNABINOID COMPOSITIONS AND METHODS OF TREATMENT

(71) Applicant: Mark J. Rosenfeld, Draper, UT (US)

(72) Inventor: Mark J. Rosenfeld, Draper, UT (US)

(21) Appl. No.: 17/216,122

(22) Filed: Mar. 29, 2021

Related U.S. Application Data
(60) Provisional application No. 63/001,450, filed on Mar. 29, 2020.

(51) Int. Cl. A61K 31/05; A61K 9/00; A61P 29/00; A61K 47/44

(52) U.S. Cl. CPC A61K 31/05; A61K 47/44; A61P 29/00; A61K 9/0053

(57) ABSTRACT

The present disclosure is drawn to oral cannabinoid compositions and methods for modulating inflammation in a subject. In some aspects, oral administration of an amount of a cannabinoid in a carrier to a subject reduces cytokine levels in the subject to a greater degree than an equivalent amount of the cannabinoid administered in an edible oil.

### PRIORITY DATA

This application claims the benefit of U.S. Provisional Application Ser. No. 63/001,450, filed on Mar. 29, 2020, which is incorporated herein by reference.

### FIELD OF THE INVENTION

The present disclosure relates to methods of modulating inflammation in a subject and treating inflammatory and other related conditions using cannabinoids. Accordingly, this invention involves the fields of chemistry, pharmaceutical sciences, medicine, and other health sciences.

### BACKGROUND

Cannabinoids are naturally found in a number of plants within the Cannabis genus, and in particular in the species Cannibis sativa, Cannabis indicia, and Cannabis ruderali. Of the known cannabinoid compounds, two compounds, namely, tetrahydrocannabinol (THC) and cannabidiol (CBD) have been most widely studied.

Cannabinoids have been used for many years in a primarily unregulated manner both for therapeutic and recreational purposes. Cannabinoid therapy has been tested for a wide number of conditions and indications, including anxiety and depression, neuropathic pain, nausea, acne, and cancer-related symptoms, among others. In 2018, the U.S. Food and Drug Administration approved an oral cannabidiol solution, now distributed under the name Epidiolex® for the treatment of seizures associated with two rare and severe forms of epilepsy, Lennox-Gastaut syndrome and Dravet syndrome, in patients two years of age and older.

While cannabinoids have shown to be potentially therapeutic for a number of ailments and conditions, one primary barrier to therapeutic implementation is the difficulty in administering cannabinoids in an efficient and effective manner through certain delivery routes. Of course, the most convenient and user-compliant administration route is the oral route of administration. Unfortunately, many cannabinoids, such as cannabidiol are highly hydrophobic and can only be negligibly absorbed through the aqueous environment of the enteral system without significant assistance.

In an attempt to boost enteral bioavailability, oral cannabinoid formulations to date have primarily consisted of cannabinoid extracts admixed with an edible oil, such as olive oil. Other attempts to boost bioavailability through the use of other formulation vehicles have also been made. However, generally speaking, bioavailability of orally administered cannabinoids remains poor.

### SUMMARY

The present disclosure is drawn to methods of modulating cytokine levels in a subject and compositions and dosage forms useful therefor. In one embodiment, a method of modulating cytokine levels in a subject can include orally administering to the subject, at least one cannabinoid in a carrier and in an amount that reduces cytokine levels in the subject to a greater degree than an equivalent amount of the cannabinoid administered in an edible oil, such as olive oil, or a cannabinoid administered from a carrier consisting essentially of, or containing only, an edible oil, such as olive oil.

In some embodiments, the subject's cytokine levels are reduced by more than 30% compared to a reduction provided by the cannabinoid administered in the edible oil. In other embodiments, the subject's cytokine levels are reduced by between about 30% and 90% as compared to a reduction provided by the cannabinoid administered in the edible oil. In yet another embodiment, the subject's cytokine levels are reduced by at least 70% as compared to a reduction provided by the cannabinoid administered in the edible oil.

In some embodiments, the cytokine is a pro-inflammatory cytokine. In other embodiments, the pro-inflammatory cytokine is a member selected from the group consisting of: chemokines (CC), interferons (IFN), interleukins (IL), lymphokines (LK), tumor necrosis factors (TNF) or a combination thereof. In yet further embodiments, the pro-inflammatory cytokine can be a tumor necrosis factor, such as a TNF-α. In other embodiments, the pro-inflammatory cytokine can be an interleukin, such as IL-6.

In additional embodiments, the cannabinoid can include cannabidiol. In yet other embodiments, the cannabinoid can include tetrahydrocannabinol (THC), such as Delta9-THC or Delta8-THC).

In further embodiments, the cannabinoid can be administered to the subject in an amount of from about 5 mg/kg of the subject's body weight to about 50 mg/kg of the subject's body weight.

In additional embodiments, the carrier can include a mixture of at least one oil and a combination of surfactants. In some further embodiments, the mixture of surfactants can include at least one hydrophilic surfactant.

In some embodiments, the amount of CBD in the carrier and the amount of CBD in the edible oil can be substantially the same. In additional embodiments, the amount of CBD in the carrier is less than the amount of CBD in the edible oil. In further embodiments, the amount of CBD in the carrier can be from at least 3 times less to 10 times less than the amount of CBD in the edible oil.

The cytokine levels in the subject can result from a cytokine dysregulation event. In one embodiment, such a dysregulation event can occur in connection with at least one of: an autoimmune condition; a virally induced condition; a bacterially induced condition; or a wound. In another embodiment, the dysregulation event can occur in connection with a SARS-CoV-2 infection.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure; and, wherein:

FIG. 1 is a graphical depiction of amounts of CBD in a carrier as disclosed herein as compared to amounts of CBD in edible oil.

FIG. 2 is a graphical representation of CBD plasma levels achieved in a subject by CBD in a carrier as disclosed herein as compared to CBD in edible oil.

FIG. 3 a graphical depiction of the impact on TNF alpha (TNF-α) serum levels in subjects who received various amounts of CBD in a carrier as recited herein, compared to amounts of CBD in edible oil, further compared to a control.

Reference will now be made to the exemplary embodiments illustrated, and specific language will be used herein to describe the same. It will nevertheless be understood that no limitation of the scope of the invention is thereby intended.

### DETAILED DESCRIPTION

Before invention embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting.

Furthermore, the described features, structures, or characteristics can be combined in any suitable manner in one or more embodiments. In the following description, numerous specific details are provided, such as examples of compositions, dosage forms, treatments, etc., to provide a thorough understanding of various invention embodiments. One skilled in the relevant art will recognize, however, that such detailed embodiments do not limit the overall inventive concepts articulated herein, but are merely representative thereof.

### Definitions

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As used herein, the terms “treat,” “treatment,” or “treating” and the like refers to administration of a therapeutic agent to a subject who is either asymptomatic or symptomatic. In other words, “treat,” “treatment,” or “treating” can refer to the act of reducing or eliminating a condition (i.e., symptoms manifested), or it can refer to prophylactic treatment (i.e., administering to a subject not manifesting symptoms in order to prevent their occurrence). Such prophylactic treatment can also be referred to as prevention of the condition, preventative action, preventative measures, and the like.

As used herein, the terms “therapeutic agent,” “active agent,” and the like can be used interchangeably and refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts. Further, when these terms are used, or when a particular active agent is specifically identified by name or category, it is understood that such recitation is intended to include the active agent per se, as well as pharmaceutically acceptable salts, esters or compounds significantly related thereto, including without limitation, prodrugs, active metabolites, isomers, and the like.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format for administration to a subject. For example, an “oral dosage form” can be suitable for administration to a subject's mouth. A “topical dosage form” can be suitable for administration to a subject's skin by rubbing, etc.

As used herein, “pharmaceutically acceptable carrier” or “carrier” are used interchangeably and refer to a pharmaceutical acceptable agent that can be capable of fully or partially dissolving or solubilizing an active agent (e.g., cannabidiol) in the pharmaceutical composition. Further, in some aspects, the carrier can be an agent that can be varied for the alteration of release rate and/or extent of the active agent from the composition and/or the dosage form.

As used herein, “cannabinoid” or “cannabinoids” refers to one or more compounds found in a Cannibis plant, such as the species of Cannibis sativa, Cannabis indicia, and Cannabis ruderali, including hemp plants, such as industrial hemp. Notable cannabinoids include tetrahydrocannabinol (THC) (Delta9-THC or Delta8-THC), and cannabidiol (CBD). Cannabidiol has the general structure:

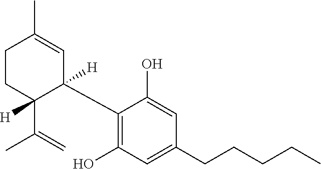 [SMILES: C=C(C)[C@@H]1CCC(C)=C[C@H]1c1c(O)cc(CCCCC)cc1O]

and is known under the IUPAC name 2-[(1R,6R)-3-methyl-6-prop-1-en-2-ylcyclohex-2-en-1-yl]-5-pentylbenzene-1,3-diol. It should be understood that recitation of the term “cannabinoid” in this written description provides express support for each and every cannabinoid species, including CBD and TCH an vice versa.

As used herein, a “semi-liquid” corresponds to a partially solubilized active agent and a “liquid” corresponds to a fully solubilized active agent.

As used herein, a “subject” refers to a mammal that may benefit from the administration of a drug composition or method disclosed herein. Examples of subjects include humans, and may also include other animals such as horses, pigs, cattle, dogs, cats, rabbits, and aquatic mammals. In one specific aspect, a subject is a human. In another aspect, the subject is a female. In another aspect, the subject is male.

As used herein, “in need of treatment” refers to a subject that has a disease or is suspected of having the disease according to various diagnostic criteria typically used in practice, or desires treatment or is indicated for treatment. Thus, “in need of treatment” can include the operation of identifying a subject in need of treatment.

As used herein, “identifying a subject in need of treatment” can include the operation of obtaining a biological sample from the subject and determining the level of one or more biomarkers as described herein, assessing a biological sample obtained from said subject, performing an imaging analysis on the subject, assessing one or more clinical characteristics of said subject (e.g., assessing symptoms or overt symptoms), or a combination thereof.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, an asthma attack, bronchitis, a heart attack, pneumonia, and the like. Some examples of chronic conditions can include without limitation, arthritis, diabetes, hypertension, high cholesterol, and the like.

As use herein with respect to physiologic levels of a given substance, the term “baseline” refers to a level or concentration of the substance in a subject prior to administration of an active agent. For example, the baseline level of pregn-4-ene-3,20-dione in a subject would the subject's pregn-4-ene-3,20-dione serum level prior (e.g., just prior) to the commencement of pregn-4-ene-3,20-dione administration or therapy.

The term “oral administration” represents any method of administration in which an active agent can be administered by swallowing, chewing, sucking, or drinking of the dosage form. Oral administration can be intended for enteral delivery of an active agent or transmucosal delivery of the active agent. In some embodiments, the composition of the current inventions can be admixed with food or drink prior to being orally consumed.

As used herein, the terms “release” and “release rate” are used interchangeably to refer to the discharge or liberation of a substance, including without limitation a drug, from the dosage form into a surrounding environment such as an aqueous medium either in vitro or in vivo.

In some aspects of the present invention, the release of the drug may be controlled release. As used herein, the term “controlled release” represents the release of the drug from the dosage form according to a predetermined profile. In some aspects, the controlled release selected can be, intermediate, delayed, extended, sustained, pulsatile, gastric, enteric or colonic. In another aspect, combinations of the aforementioned release profiles may be used in order to achieve specific delivery results, such as an immediate release followed by a delayed and/or a sustained release of the active agent.

As used herein, a “dosing regimen” or “regimen” such as an “initial dosing regimen” or “starting dose” or a “maintenance dosing regimen” refers to how, when, how much, and for how long a dose of the compositions of the present invention can be administered to a subject. For example, an initial or starting dose regimen for a subject may provide for a total daily dose of from about 5 mg/kg of body weight to about 50 mg/kg of body weight.

As used herein, “daily dose” refers to the amount of active agent (e.g., cannabidiol) administered to a subject over a 24-hour period of time. The daily dose can be administered one or more administrations during the 24-hour period. In one embodiment, the daily dose provides for two administrations or more in a 24-hour period.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or by other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein “single unit” when used to describe dosing of a subject refers to the dosage form being a single dosage form, e.g. a single tablet, capsule, pump or squirt of gel or solution, etc. In contrast, “multiple unit” when used to describe dosing of a subject refers to the dosage including two or more dosage forms, e.g. 2 capsules, 3 tablets, 2-4 pumps or squirts, etc. It is noteworthy that multiple unit dosage forms generally will be the same type of dosage forms (i.e. tablet or capsule) but are not required to be the same dosage form type.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, a composition that “increases” CBD serum levels provides a CBD level in a subject that is elevated as compared to a serum level at a previous point in time, such as a baseline level (e.g., prior to treatment), or as compared to an earlier treatment with a different (e.g., lower dose).

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Description

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, variants, modifications, and equivalents as may be included within the spirit and scope of the invention as defined by the appended claims.

An initial overview of technology embodiments is provided below, and then specific technology embodiments are described in further detail later. This initial summary is intended to aid readers in understanding the technology more quickly but is not intended to identify key features or essential features of the technology nor is it intended to limit the scope of the claimed subject matter.

At first glance, COVID-19 virus infections seem to have little in common with adverse health conditions like psoriasis, inflammatory bowel disease, rheumatoid arthritis and even diabetes. But an underlying pathological mechanism common to all of these involves one or more cytokine proteins, such as Tumor Necrosis Factor Alpha, along with an over-reactive immune response may allow a molecule that can treat these adverse health conditions, cannabidiol (CBD), to save the lives of coronavirus victims.

A “cytokine storm” refers to a life-threatening hyperinflammatory state caused by rapidly proliferating, highly activated T-cells or natural killer (NK) cells and the consequent excessive release of proteinaceous inflammatory mediators called cytokines. Both pro-inflammatory cytokines, like Tumor Necrosis Factor Alpha (TNF-α), Interleukin-1 (IL-1) and Interleukin-6 (IL-6), and anti-inflammatory cytokines, such as Interleukin 10 (IL-10), excessively accrue when a cytokine storm occurs. A fierce and oftentimes deadly interplay then occurs between these molecules leading to life-threatening complications like pulmonary embolism and pulmonary edema.

As with other coronaviruses, a COVID-19 infection turns deadly when it progresses to a “cytokine storm”. Almost invariably the cause of death from a COVID-19 infection, it manifests as severe respiratory distress and is accompanied by the shutdown of multiple organs. Since a life-threatening cytokine storm results from a COVID-19 infection with disconcerting frequency, effective therapies have been deemed key to patient survival. Anti-inflammatory and immunosuppressive drugs have not been successful in treating a cytokine storm and thus improving survival. Nonsteroidal anti-inflammatory drugs (NSAIDS) can alleviate lesser infections, but not a cytokine storm. Corticosteroids are problematic. These complicate already life-threatening circumstances due to the increased risk of hospital-acquired infections and mortalities otherwise associated with their use. Anti-TNF-α and anti-IL-6 receptor monoclonal antibodies exist, but their use comes with the prospect of worsening the cytokine storm itself. A bottom line is that feasible treatment regimens remain tragically lacking. But these are mandatory for successfully treating COVID-19, influenza and other viral infections.

CBD, a natural, non-intoxicating substance obtained from industrial hemp (Cannabis sativa), suppresses the production of several cytokines, thus making it a putative immunomodulatory therapeutic. Importantly, this attribute is complemented by CBD's eminently desirable safety profile. It is well tolerated when introduced into the body, and limited risk is otherwise associated with use. Such safety offers distinct advantages over other pharmaceuticals. Adverse effects principally center about CBD's interactions with other medications.

Tumor necrosis factor alpha (TNF alpha or TNF-α) is a major driver of immune-mediated diseases, concerning which predominately anti-TNF-α monoclonal antibodies, but also other anti-TNF approaches have figured prominently in their therapy. However, an elevated risk of serious infections comes with monoclonal antibody use, and this is further complicated by additional side effects. These problems are roadblocks using anti-TNF-α monoclonal antibodies with COVID-19 infections. It would seem that these complications could confound an already life-threatening circumstance. Nevertheless, restricting TNF-α warrants serious consideration, because TNF-α is a primary driver of deadly cytokine storms as well as sepsis and cytokine release syndrome.

One overriding issue with CBD therapy is CBD's poor oral bioavailability. Bioavailability refers to how much of an oral dose reaches the bloodstream and thus is able to reach sites of action in the body. How well a drug achieves a desired effect directly relates to bioavailability. Higher bioavailabilities are important for achieving consistent and effective pharmacological activity.

The oral bioavailability of CBD in oil, such as edible oil, the typical mode of oral administration, in humans is about 6%, much less than the 20-30% typically needed for a drug to achieve consistent therapeutic results.

This low bioavailability makes it difficult to efficiently bring CBD into the bloodstream. It cannot sidestep degradation in gastrointestinal acid, is unable to readily dissolve in the aqueous environment of the gut lumen and cannot readily transit the wall of the small intestine. CBD needs serious assistance to efficiently surmount these hurdles and, without help, CBD cannot become useful medicine.

The present formulations and methods overcome this problem by delivering CBD in a carrier that dramatically improves bioavailability. In such a carrier, CBD is ensconced in stable vessels of exceptionally small size (15 nm diameter) made with GRAS substances and bioavailability is enhanced. GRAS is the acronym for “generally recognized as safe, the USFDA designation that a substance added to food is deemed safe by experts, and so is exempted from Food, Drug, and Cosmetic Act food additive tolerance requirements.

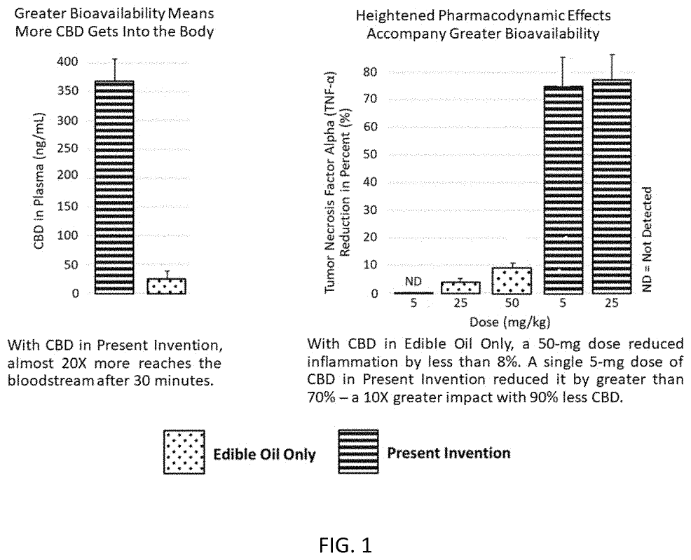

Referring to FIG. 1, there is shown a comparison of CBD serum levels results obtained by the present CBD compositions and methods as compared to the same amount of CBD in an edible oil. As can be seen, the serum levels of CBD obtained by the present CBD compositions and methods range between 300 ng/ml and 420 ng/ml after 30 minutes of oral administration, while the CBD in an edible oil composition containing the same amount of CBD ranged between 10 ng/ml and 40 ng/ml within the same time period. This represents an almost 20 fold increase in bioavailability for the present CBD compositions and methods as compared to the formulation of CBD in an edible oil.

Referring again to FIG. 1, is shown a comparison of the reduction in inflammation achieved by the present CBD compositions and methods as compared to CBD in an edible oil. As can be seen, the amount of TNF-α reduction seen for the present formulations and methods containing 5 mg of CBD or 25 mg of CBD was more than 70% greater than the reduction of inflammation achieved by comparable CBD in an edible oil formulations. In fact, in some embodiments, the amount of inflammation reduction obtained with the present CBD formulations and methods is 60% greater than a comparable CBD in oil formulation having 10 times the amount of CBD.

In some embodiments, the present CBD compositions and methods can reduce cytokine levels by more than 30% compared to a reduction provided by the cannabinoid administered in the an edible oil. In further embodiments, the cytokine levels are reduced by between about 30% and 90% as compared to a reduction provided by the cannabinoid administered in an edible oil. In yet further embodiments, the cytokine levels are reduced by at least 70% as compared to a reduction provided by the cannabinoid administered in an edible oil.

In further embodiments, the cytokine that is reduced can be a pro-inflammatory cytokine, an anti-inflammatory cytokine, or a combination thereof. For example pro-inflammatory cytokines can include chemokines (CC), interferons (IFN), interleukins (IL), lymphokines (LK), tumor necrosis factors (TNF) or a combination thereof. In one embodiment, the cytokine is a tumor necrosis factor. In another embodiment, the tumor necrosis factor is TNF alpha (TNF-α). In a further embodiment, the pro-inflammatory cytokine is an interleukin. In another embodiment, the interleukin is IL-6.

The CBD formulations and methods recited herein manifest the ability to considerably reduce TNF-α levels prodigiously more than does CBD in oil, the usual way in which CBD is consumed. As a primary driver of cytokine storms, being able to substantively reduce the presence of TNF-α is a strong indicator that such compositions and methods have the potential to be lifesaving mitigators or prophylactic protectors against cytokine dysregulation conditions, such as cytokine storms, sepsis and cytokine release syndrome.

In one case study, the present CBD formulations and methods were tested on a condition in which TNF-α plays a major role, chronic plaque psoriasis. Patients were rendered virtually symptom free with present CBD formulations and methods, something never achieved with therapies other than monoclonal antibodies to the best of our knowledge. It outperformed monoclonal antibodies in the sense that patients were symptom free in less than a month rather than one year.

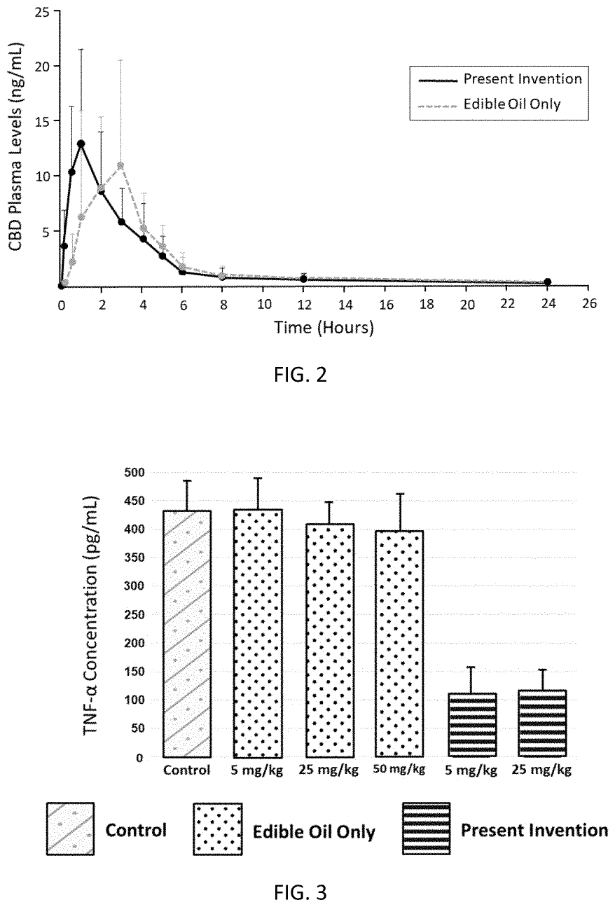

Complementing the above results is an FDA-compliant Phase 1 human pharmacokinetic clinical trial done at Hadassah Hospital in Jerusalem, Israel (FIG. 2). As can be seen from FIG. 2, the trial confirms that present CBD compositions and methods rapidly transfer CBD into the bloodstream. At 30 minutes post ingestion, approximately 15 times more CBD gets into the body with a 50-mg dose. Furthermore, Tmax (the time after administration when the maximum plasma concentration is reached) for it was 1 hour instead of the 2 or more hours needed with CBD in an edible oil, such as an edible oil.

With COVID-19, the transition to uncontrolled cytokine release can rapidly occur. So, drugs to counteract this potentially deadly state must likewise quickly work. The pharmacokinetic results above discussed indicate that present compositions and methods CBD can do this.

Turning further to the issue of TNF-α reduction, it has been established that TNF-α is one of the most important cytokines made by macrophages in the sense that it prominently figures in many diseases of conditions. For reducing TNF-α, CBD formulations and methods of the present invention considerably outperformed CBD in an edible oil, the overwhelming mode of oral CBD administration worldwide. In that regard, CBD in invention must be considered as a treatment in the many circumstances where reducing levels of TNF-α levels is desirable.

TNF-α figures importantly in the etiology of both type 1 and type 2 diabetes mellitus, which means that the present formulations and methods could play a role in their treatments.

TNF-α drives a low-grade inflammatory condition that precedes and accompanies both sorts of disease. TNF-α also plays a role in painful peripheral, autonomic, proximal and focal neuropathies associated with type 2 diabetes. It suppresses insulin transmembrane signaling, which leads to the insulin resistance that typifies type 2 disease. Insulin resistance means that cells in the body cannot properly retrieve glucose from the blood. Cells use glucose as an essential energy source, and insulin is the hormone necessary for moving glucose from the blood into cells. TNF-α also turns off adiponectin production in adipocytes, the cells that make up adipose or fat tissue. Adiponectin likewise plays a role in regulating glucose levels, and its decreased presence further drives insulin resistance. The immunosuppression of TNF-α by the present formulations and methods may be enough to successfully remedy such circumstances.

The present formulations and methods also have a potential role in treating type 1 diabetes, by preventing the pancreatic β-cell death driven by TNF-α. This cytokine is prominently elevated during progression of this disease, where it plays an active role in the autoimmunity responsible for pancreatic β-cell destruction. Antigens are substances that cause an immune response. TNF-α accelerates the maturation of dendritic cells, antigen-presenting cells whose main function is to carry antigens to T cells, immune cells that protect the body from pathogens and abnormal cell growth. In type 1 diabetes, matured dendritic cells cause activation of the islet-specific pancreatic lymph node T cells that cause pancreatic β-cell damage and death. Such activity could eventually result in an irreversible condition in which the pancreas produces no insulin, because its β-cells have been eradicated. At that point, individuals must depend on daily insulin injections or an insulin pump to control their blood glucose levels.

The impressive immunosuppressive capacity experienced with the present formulations and methods might thus be exploited as a type 1 diabetes therapeutic during the honeymoon phase of this disease while β-cells still exist. The honeymoon phase denotes the time span after type 1 diabetes diagnosis when the pancreas can still manufacture enough insulin to assist in blood glucose control. Type 1 diabetics are typically diagnosed during the honeymoon phase. It can last up to two years post diagnosis, during which the pancreas makes progressively less new β-cells and insulin as older ones die. Eventually exogenous insulin dependency becomes total and irrevocable. TNF-α conspicuously figures in the demise of pancreatic β-cells, which makes reducing its presence desirable for delaying or even preventing complete insulin dependency, or perhaps for even reversing type 1 disease.

It is herein further suggested that the present compositions and methods could be applied as a complement to the allogeneic transplantation of healthy β-cells being pursued for regaining endogenous insulin production, let alone organ transplants of other sorts, in the sense that it might help to control graft-versus-host-disease.

TNF-α is also key to the progression of the osteoarthritis seen in type 2 diabetics, let alone osteoarthritis in general. While insulin plays a protective, anti-inflammatory role in the synovium, insulin resistance in patients with type 2 disease may impair this protective effect and promote the development of arthritic disease. Due to its anti-TNF-α activity, the present compositions and methods could be a tool for reversing or constraining osteoarthritis associated with diabetes along with diabetes itself.

Cardioavacular disorders are additional disease states that might benefit from the present compositions and methods given not only their considerable immunosuppressive capacity, but also its potentially rapid acting nature. TNF-α in excess drives these conditions. It is a major factor in heart failure, where rapidly decreasing its presence could assist in prolonging life, and TNF-α may also prove desirable for treating cardiac infarction, pulmonary arterial hypertension, aortic inflammation, peripheral artery disease, atherosclerosis, and more.

CBD has been under growing study in academic, medical and commercial settings regarding its potential to treat many clinical indications. In that regard, there has been wide active dosing range for it that sometimes seemed prodigious and perhaps excessive. The therapeutic potential of the present compositions and methods appears considerable. As a result thereof, a smaller amount of CBD may be needed to achieve a therapeutic effect, which could portend reductions in side effects like idiopathic liver disease and drug-drug interactions. The present compositions and methods not only have a considerable capacity as to reducing TNF-α, but may also be able to do this in short order. So, rapid action could have life-saving potential where TNF-α is driving deadly situations.

The present compositions or formulations can include a cannabinoid, such as cannabidiol (CBD) and a carrier, for example, a carrier that includes more than just an edible oil, such as olive oil. In one embodiment, the cannabinoid can be present in the carrier and in an amount that reduces cytokine levels in the subject to a greater degree than an equivalent amount of the cannabinoid administered in an edible oil.

In some embodiments, the cannabinoid can be cannabidiol (CBD), cannabidiolic acid (CDBA), or tetrahydrocannabinol (THC), or combinations thereof.

In some embodiments, the cannabinoid can be present in the compositions in amounts ranging from 0.0001 wt % to 85 wt % of the composition. In some embodiments, the cannabinoid can be present in an amount of from about 5 wt % to about 75 wt %. In yet another embodiment, the cannabinoid can be present in an amount of from about 10 wt % to about 50 wt %. In other embodiments, the cannabinoid can be present in an amount of from about 2 wt % to about 10%.

The carrier can include a number of ingredient that contribute to or otherwise achieve the increased or enhanced bioavailability as compared to a composition of CBD in an edible oil. Such ingredients can generally include surfactants, including hydrophilic, hydrophobic, and amphoteric surfactants, solubilizers, solvents, cosolvents, and additives, as well as oils, fats, carotenoids, nanocarotinoids, among others.

A wide variety of solubilizers can be used in the carrier of the present compositions. Examples of suitable solubilizers can comprise but are not limited to: (i) tocopherol or its derivatives; (ii) fatty acid or its salts; (iii) glyceryl fatty acid esters; (iv) PEG glycerides of fatty acid esters; (v) polyglycerol fatty acid esters; (vi) triglycerides; (vii) hydrogenated polyoxyl vegetable oils or glycerides; (viii) propylene glycol fatty acid esters; (ix) vegetable oils; (x) sterols or its derivatives, the like, or combinations thereof.

In one aspect, vitamin E or its derivatives can comprise, but are not limited to: alpha-tocopherol, beta-tocopherol, gamma-tocopherol, delta-tocopherol, tocopherol acetate, tocopherol linoleate, tocopherol succinate, tocotrienols (alpha-, beta-, gamma-, or delta-), tocofersolan (PEG derivatives of alpha-tocopherol), the like, or combinations thereof.

In another aspect, fatty acid or its salts can comprise, but not limited to: octanoic acid, capric acid, lauric acid, myristic acid, palmitic acid, stearic acid, oleic acid, linoleic acid, linoelaidic acid, sodium caproate, sodium caprylate, sodium laurate, sodium myristate, sodium palmitate, sodium oleate, sodium stearate, sodium lauryl sulfate, sodium lauryl sarcosinate, sodium dioctyl sulfosuccinate, sodium cholate, sodium taurocholate, the like, or combinations thereof.

In another aspect, glyceryl fatty acid esters can comprise, but are not limited to: glyceryl monooleate, glyceryl monoleate/linoleate, glyceryl monolinoleate, glyceryl ricinoloeate, glyceryl monolaurate, glyceryl monopalmitate, glyceryl monostearate, glyceryl mono-/di-oleate, glyceryl palmtate/stearate, glyceryl acetate, glyceryl laurate, glyceryl citrate/lactate/oleate/linoleate, glyceryl caprylate, glyceryl caprylate/caprate, glyceryl dicaprylate/dicaprate, mono-/di-acetylated monoglycerides, glyceryl monostearate, glyceryl dilaurate, glyceryl dioleate, the like, or combinations thereof.

In another aspect, PEG glycerides of fatty acid esters can comprise, but are not limited to: PEG fatty acid monoesters, PEG glycerol fatty acid esters, PEG fatty acid diesters, PEG fatty acid mono-/di-ester mixtures, PEG triglycerides of fatty acid esters, the like, or combinations thereof. PEG glycerol fatty acid esters can comprise, but are not limited to: PEG glyceryl laurate, PEG glyceryl laurate, PEG glyceryl caprylate, PEG glyceryl caprate, PEG glyceryl oleate, PEG glyceryl mono-/di-fatty acid ester mixtures, the like, or combinations thereof. PEG fatty acid monoesters can comprise, but are not limited to: esters of caprylic acid, capric acid, lauric acid, oleic acid, and stearic acid, the like, or combinations thereof. Examples of the PEG fatty acid monoesters can include PEG (1-100, 200, 300, 400) monocaprylate, PEG (1-100, 200, 300, 400) monocaprate, PEG (1-100, 200, 300, 400) monolaurate, PEG (1-100, 200, 300, 400) monooleate, PEG (1-100, 200, 300, 400) monopalmitate, PEG (1-100, 200, 300, 400) monostearate, and PEG (1-100, 200, 300, 400) monococoate, the like, or combinations thereof. PEG fatty acid diesters can comprise, but are not limited to, PEG (4-32) dicaprylate, PEG (4-32) dicaprate, PEG (4-32) dilaurate, PEG (4-32) dioleate, PEG (4-32) distearate, and PEG (4-32) dipalmitate, the like, or combinations thereof. PEG fatty acid mono-/di-ester mixtures can comprise, but are not limited to: PEG caprylate/caprate, PEG mono-/di-caprylate, PEG mono-/di-caprate, PEG mono-/di-laurate, PEG mono-/di-oleate, and PEG mono-/di-stearate the like, or combinations thereof. PEG triglycerides of fatty acid esters can comprise, but are not limited to: lauroyl polyoxylglycerides, stearoyl polyoxylglycerides, oleoyl polyoxyl glycerides, linoleoyl polyoxyl glycerides, lauroyl polyoxyl glycerides, caprylocaproyl polyoxyl glycerides, and behenoyl polyoxylglycerides the like, or combinations thereof.

In yet another aspect, polyglycerol fatty acid esters can comprise, but are not limited to: polyglyceryl (2, 3, 4, 6, 10) oleate, polyglyceryl (2, 3, 4, 6, 10) dioleate, polyglyceryl (2, 3, 4, 6, 10) trioleate, polyglyceryl (2, 3, 4, 6, 10) laurate, polyglyceryl (2, 3, 4, 6, 10) dilaurate, polyglyceryl (2, 3, 4, 6, 10) trilaurate, polyglyceryl (2, 3, 4, 6, 10) stearate, polyglyceryl (2, 3, 4, 6, 10) distearate, polyglyceryl (2, 3, 4, 6, 10) tristearate, polyglyceryl (2, 3, 4, 6, 10) mono-/di-oleate, polyglyceryl (3,6,10) caprate, polyglyceryl (3,6,10) dicaprate, polyglyceryl (3,6,10) tricaprate, polyglyceryl (3,6,10) caprylate, polyglyceryl (3,6,10) dicaprylate, polyglyceryl (3,6,10) tricaprylate, polyglyceryl (3,6,10) polystearate, polyglyceryl (3,6,10) polyoleate, polyglyceryl (3,6,10) mono-/di-oleate, polyglyceryl (3,6,10) caprylate, polyglyceryl (3,6,10) polycaprylate, polyglyceryl (3,6,10) caprate, polyglyceryl (3,6,10) polycaprate, and polyglyceryl (3,6,10) caprylate/caprate, the like, or combinations thereof.

In another aspect, triglycerides can comprise, but are not limited to: glyceryl tricaprylate, glyceryl tricaprate, glyceryl tricaprylate/tricaprate, glyceryl tricaprylate/tricaprate/trisuccinate, glyceryl trioleate, glyceryl tristearate, glyceryl trilaurate, medium chain natural oils, the like, or combinations thereof.

In yet another aspect, hydrogenated polyoxyl vegetable oils or glycerides can comprise, but are not limited to: castor oil or hydrogenated castor oil, or an edible oil (e.g. vegetable oil) such as corn oil, olive oil, peanut oil, palm kernel oil, apricot kernel oil, peppermint oil, coconut oil, sunflower seed oil, or almond oil, the like, or combinations thereof. canola oil, cotton oil, palmolein, sunflower oil, corn oil, rapeseed oil, grape seeds oil, hemp oil, pomegranate oil, avocado oil, peppermint oil and medium chain triglycerides. The polyoxyl group can include glycerol, propylene glycol, ethylene glycol, polyethylene glycol, sorbitol,pentaerythritol, the like, or combinations thereof. c Examples of hydrogenated polyoxyl vegetable oils or glycerides can comprise, but are not limited to: PEG-35 castor oil (Incrocas-35, Koliphor EL), PEG-40 hydrogenated castor oil (Koliphor RH 40), PEG-25 trioleate (TAGATRTO), PEG-60 cornglycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylickcapric glycerides (Labrasol), PEG-6 caprylic/capric glycerides (Softigen 767), PEG-5 hydrogenated castor oil, PEG-7 hydrogenated castor oil, PEG-9 hydrogenated castor oil, PEG-6 corn oil (Labrafil M. 2125 CS), PEG-6 almond oil (Labrafil M 1966 CS), PEG-6 apricot kernel oil (Labrafil M 1944CS), PEG-6 olive oil (Labrafil M 1980 CS), PEG-6 peanut oil (Labrafil M 1969 CS), PEG-6 hydrogenated palm kernel oil (Labrafil M2130 BS), PEG-6 palm kernel oil (Labrafil M 2130 CS), PEG-6 triolein (Labrafil M 2735 CS), PEG-8 corn oil (Labrafil WL 2609 BS), PEG-20 corn glycerides (Crovol M40), and PEG-20 almond glycerides (Crovol A40), the like, or combinations thereof.

The oil may be present in the formulation, according to some embodiments, at an amount of between about 0.5 and 20 wt %. According to other embodiments, the oil is present in the formulation at an amount of between about 1 and 10 wt %.

In one aspect, propylene glycol fatty acid esters can comprise, but are not limited to: propylene glycol monolaurate (Lauroglycol FCC), propylene glycol ricinoleate (Propymuls), propylene glycol monooleate (Myverol P-06), propylene glycol dicaprylate/dicaprate (Captex 200), and propylene glycol dioctanoate (Captex 800), propylene glycol monocaprylate (Capryol 90, Nikkol Sefsol 218), propylene glycol myristate, propylene glycol monostearate, propylene glycol ricinolate, propylene glycol isostearate, propylene glycol caprylate/caprate, propylene glycol dioleate, propylene glycol distearate, propylene glycol dilaurate, propylene glycol dicaprylate, and propylene glycol dicaprate, the like, or combinations thereof.

In another aspect, vegetable oils can comprise, but are not limited to, corn oil, olive oil, peanut oil, coconut oil, peppermint oil, sunflower seed oil, castor oil, safflower oil, borage oil, cottonseed oil, soybean oil, palm kernel oil, apricot kernel oil, almond oil, the like, or combinations thereof.

In one aspect, sterols or its derivatives can comprise, but are not limited to: cholesterol, sitosterol, lanosterol, phytosterol, its PEG derivatives, the like, or combinations thereof.

In one embodiment, an additive can be substance that can be added to the pharmaceutical formulation to enhance the solubilization, separation, or dispersion of the particles, or to enhance the dissolution and further absorption of the particles into the body. As used herein an additive can be a lipophilic additive when it has an HLB value of 10 or less, or a hydrophilic additive when it has an HLB value of greater than 10.

In one aspect, the pharmaceutically acceptable carrier can comprise a hydrophilic additive, a lipophilic additive, or a combination thereof. In one aspect, lipophilic additives can comprise, but are not limited to: mono-, di-glycerides of fatty acids, reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (e.g. Labrafil M 2125 CS), PEG-6 almond oil (e.g. Labrafil M 1966 CS), PEG-6 apricot kernel oil (e.g. Labrafil M 1944 CS), PEG-6 olive oil (e.g.Labrafil M 1980 CS), PEG-6 peanut oil (e.g. Labrafil M 1969 CS), PEG-6 hydrogenated palm kernel oil (e.g. Labrafil M 2130 BS), PEG-6 palm kernel oil (e.g. Labrafil M 2130 CS), PEG-6 triolein (e.g. Labrafil M 2735 CS), PEG-8 corn oil (e.g. Labrafil WL 2609 BS), PEG-20 corn glycerides (e.g. Crovol M40), PEG-20 almond glycerides (e.g. Crovol A40), lipophilic polyoxyethylene-polyoxypropylene block co-polymers (e.g. Pluronic L92, L101, L121 etc.), propylene glycol fatty acid esters, such as propylene glycol monolaurate (e.g. Lauroglycol FCC), propylene glycol ricinoleate (e.g. Propymuls), propylene glycol monooleate (e.g. Myverol P-06), propylene glycol dicaprylate/dicaprate (e.g. Captex® 200), and propylene glycol dioctanoate (e.g. Captex® 800), propylene glycol mono-caprylate (e.g. Capryol® 90); propylene glycol oleate (e.g. Lutrol OP2000); propylene glycol myristate; propylene glycol mono stearate; propylene glycol hydroxy stearate; propylene glycol ricinoleate; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g. Arlacel® 186); sterol and sterol derivatives such as cholesterol, sitosterol, phytosterol, phytosterol fatty acid esters, PEG-5 soya sterol, PEG-10 soya sterol, PEG-20 soya sterol, and the like; glyceryl palmitostearate, glyceryl stearate, glyceryl distearate, glyceryl monostearate, or a combination thereof sorbitan fatty acid esters such as sorbitan monolaurate (e.g. Arlacel 20), sorbitan monopalmitate (e.g. Span-40), sorbitan monooleate (e.g. Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, myristic acid, menthol, menthol derivatives, lecithin, phosphatidyl choline, bile salts, and the like, and mixtures thereof. In some cases, a solubilizer for the compositions and oral dosage forms can be a lipophilic surfactant.

The pharmaceutically acceptable carrier can also comprise a hydrophilic additive. In one aspect, the hydrophilic additive can comprise non-ionic surfactants, ionic surfactants, zwitterionic surfactants, the like, or combinations thereof. Suitable Hydrophilic surfactants can include, but are not limited to: alcohol-oil transesterification products; polyoxyethylene hydrogenated vegetable oils; polyoxyethylene vegetable oils; alkyl sulphate salts, dioctyl sulfosuccinate salts; polyethylene glycol fatty acids esters; polyethylene glycol fatty acids mono- and di-ester mixtures; polysorbates, polyethylene glycol derivatives of tocopherol, the like, or combinations thereof. Two or more hydrophilic additives from the same or different classes can be referred to as the hydrophilic surfactant unless explicitly specified. In one aspect, non-limiting examples of hydrophilic surfactants can comprise PEG-8 caprylic/capric glycerides, lauroyl macrogol-32 glyceride, stearoyl macrogol glyceride, PEG-40 hydrogenated castor oil, PEG-35 castor oil, sodium lauryl sulfate, sodium dioctyl sulfosuccinate, polyethylene glycol fatty acids mono- and di-ester mixtures, polysorbate 80, polysorbate 20, polyethylene glycol 1000 tocopherol succinate, phytosterols, phytosterol fatty acid esters, the like, or combinations thereof.

In yet another aspect, additives can comprise sterols and derivatives of sterols. In various aspects, these additional agents can be hydrophilic or lipophilic. Examples of hydrophilic sterols include but are not limited to: lanosterol PEG-24 cholesterol ether (e.g. Solulan C-24, Amerchol), PEG-30 soya sterol (e.g. Nikkol BPS-30, from Nikko), PEG-25 phyto sterol (e.g. Nikkol BPSH-25 from Nikko), PEG-30 cholestanol (e.g. Nikkol DHC, from Nikko). Examples of Lipophilic Sterol Surfactants are Cholesterol, sitosterol, Phytosterol (e.g. GENEROL series from Henkel), PEG-5 soya sterol (e.g. Nikkol BPS-S, from Nikko), PEG-10 soya sterol (e.g. Nikkol BPS-10 from Nikko), PEG-20 soya sterol (e.g. Nikkol BPS-20 from Nikko), the like, or combinations thereof.

In another aspect, the oral compositions can further comprise a polymeric release modifier. The polymeric release modifier can comprise, but is not limited to: celluloses, such as hydroxypropyl celluloses low molecular weight, low viscosity types (e.g. Methocel E5, E6, E10 E15, LV100 etc. grades), hydroxypropyl celluloses having higher molecular weight, medium to high viscosity (e.g. Methocel K4M, K15M, K100M etc), polyvinylpyrrolidones (e.g. Kollidon k17, K30 etc), polyvinyl acetates, hydroxypropyl methylcellulose (HPMC), hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, Sodium croscarmellose, carrageenan, hydroxyethyl cellulose (HEC), carboxymethyl cellulose (CMC) and carbox cellulose (CEC), sodium alginate, polycarbophil, gelatin, Xanthan gum, and sodium starch glycolate, the like, or combinations thereof.

The pharmaceutically acceptable carrier can be combined with additives in various amount ranges. For example, the lipophilic additive and hydrophilic additive can be present in amounts such that the ratio in wt % of lipophilic additive to amount of hydrophilic additive is from 1:10 to 10:1. In some embodiments, the amount can be a ratio of than 2:1 to 1:2. In another aspect the amount can be a ratio of 1:5 to 5:1.

In certain examples, the hydrophilic additive can make up about 1% to about 99% w/w, about 2% to about 80% w/w, about 2% to about 50% w/w, or about 10% to about 40% w/w of any pharmaceutical composition described herein. In some examples, the lipophilic additive can make up about 1% w/w to about 99% w/w, about 2% to about 80% w/w, about 10% w/w to about 80% w/w, about 30% w/w, to about 80% w/w, or about 40% to about 80% w/w of any pharmaceutical composition described herein.

Co-solvents can fully or partially solubilize cannabinoids when presented in an effective amount. Examples of suitable co-solvents can comprise without limitation: alcohols and polyols, such as ethanol, propanol, isopropanol, butanol, benzyl alcohol, ethylene glycol, propylene glycol, butanediols, glycerin or its derivatives thereof, glycerol, diglycerol, polyglycerol, pentaerythritol, sorbitol, mannitol, transcutol, dimethyl isosorbide, triacetin, trimethyl citrate, polyethylene glycol, polypropylene glycol, polyvinylalcohol, hydroxypropyl methylcellulose and other cellulose derivatives, cyclodextrins or its derivatives, the like, or combinations thereof.

In one aspect, the composition can be formulated as a solution, an emulsion, a liquid, a semi-liquid, a suspension, the like, or combinations thereof. In one example, the composition can form a dispersion, an emulsion, a solution, or a micellar solution upon 10× or 100× dilution with an aqueous bile salt comprising simulated gastric or intestinal media or water. In another example, the composition can remain substantially solubilized upon 10× or 100× dilution with an aqueous bile salt comprising simulated gastric or intestinal media or water. In another example, the composition can form a dispersion upon 10× or 100× dilution with an aqueous bile salt comprising simulated gastric or intestinal media or water with a mean dispersion particle size of less than 300 nm or a UV absorbance of less than 3 units when measured at 400 nm. In one aspect, the CBD can include a crystalline form that is milled, nanosized, micronized, ultra-micronized, the like, or combinations thereof.

In some embodiments there are provided methods of treatment utilizing oral cannabinoid administration to a subject. In some examples, such a method of treatment can include modulating cytokine levels in the subject. In other examples, such a method can include treating a condition or illness resulting in cytokine dysregulation, for example, autoimmune conditions or disorders, inflammatory conditions or disorders, viral infections, bacterial infections, diabetes, wounds, including surgically created wounds, psoriasis, inflammatory bowel disease and rheumatoid as well as psoriatic arthritis. Other relevant adverse conditions include demyelinating neurological disorders associated with an excess of TNF-α and other cytokines, such as multiple sclerosis, optic neuritis, Guillain-Barré syndrome, chronic inflammatory demyelinating polyneuropathy, and additional central and/or peripheral demyelinating neuropathies. TNF-α in excess is likewise a major driving factor in cardiovascular disorders like coronary artery disease and congestive heart failure.

### Examples

The following example is provided to promote a more clear understanding of certain embodiments of the present invention, and are in no way meant as a limitation thereon.

This example relates to a method for lessening in vivo TNF-α levels, said method comprising the administration of single dose of a select amount of the compounds of the invention and then an examination of the resulting TNF-α levels in a delayed type hypersensitivity inflammation (DTH) model. DTH reactions are caused by the release of mediators from activated T cells, which in turn activate local endothelial cells and cause swelling along with inflammation. This inflammation entails the production of cytokines that includes TNF-α.

Mice. Eight-week-old female outbred Sabra mice were acclimated in their cages for one week after their arrival in the laboratory. The Sabra strain was developed about 35 years ago and is widely used for research, especially in Israel. Before and during the study, all animals were housed in a controlled environment (12-12 h light-dark cycle and 24±1° C.) Food (Teklad Global Rodent Diet 2018S, 18% protein, Envigo, Israel) and water were provided ad libitum.

DTH Induction. DTH was induced in all mice by means of a sensitization-rechallenge method48. A total of 35 mice were used. Animals were randomly assigned to seven test groups (n=5 in each group) and then sensitized with a subcutaneous injection (100 μL/hind flank) of 1.5 mg/mL methylated bovine serum albumin (mBSA, Sigma Aldrich, USA) emulsified in complete Freund's adjuvant (CFA, Sigma Aldrich, USA). Six days later, all were rechallenged with a footpad injection (20 μL/footpad) of 10 mg/mL mBSA in 1× phosphate buffered saline (PBS).

CBD Preparation. CBD in invention was constructed under cGMP conditions. CBD in invention is an amphiphilic fluid composed not only of natural CBD refined from industrial hemp (99+% purity; GennCanna, Winchester, Ky., USA) but also phosphatidylcholine, polyoxyethane sorbitan monooleate, propyl glycol, polyoxyethylated castor oil and medium chain triglycerides. The same CBD used to make CBD in Invention was used for CBD in extra virgin food-grade olive oil.

CBD Administration. Following the rechallenge “footpad” injection, the animals were fasted overnight. Then, each mouse was weighed to establish the dosing regimen. CBD, either CBD in Invention or CBD in an edible oil, both appropriately diluted or suspended with PBS, and just its PBS carrier without CBD, a comparator or control, were administered via gavage as shown in the schedule immediately below. Five animals were in each treatment group:

TABLE 1

| | CBD Dose |
|---|---|
| | mg/kg |
| Treatment | Body Weight |
| PBS - Control | 0 |
| CBD in an Edible Oil | 5 |
| CBD in an Edible Oil | 25 |
| CBD in an Edible Oil | 50 |
| CBD in the Present Invention | 5 |
| CBD in the Present Invention | 25 |

Table 1. Dosing Regimens for CBD. Both CBD in invention and CBD in an edible oil were administered via gavage to mice. Doses ranged from 5 mg/kg CBD to 50 mg/kg CBD. A comparisonal or control group received only PBS, the diluent used for CBD whether in invention or in an edible oil.

Blood Sampling and Processing. Forty-eight hours after dosing with CBD or with PBS only, samples were taken for determining TNF-α in circulating blood. Prior to drawing blood, animals were first anesthetized with isoflurane, or halogenated ether (SigmaAldrich, Saint Louis, Mo., USA), was administered in a closed container49. Isofluorane is a preferred and humane, rapid-acting inhalant anesthetic for rodents.

About 1-mL blood was taken from each mouse via cardiac puncture using a 1 mL tuberculin syringe and 21-gauge needle (Becton Dickinson, Franklin Lakes, N.J., USA), immediately transferred to a 1.5-mL polypropylene microcentrifuge tube (Cole-Parmer, Vernon Hills, Ill.) and then placed on ice, where blood was permitted to clot for approximately 30 min. Samples were centrifuged at 1500 g for 15 min at 4° C. The serum fraction was then carefully removed with a sterile, plugged pipette tip, transferred to a new 1.5 mL microcentrifuge tube and placed in storage at −80° C.

TNF-α Quantification. Serum TNF-α was measured using a Mouse High Sensitivity TNF-α Solid-Phase Sandwich High Sensitivity Enzyme-Linked Immunosorbent Assay (ELISA; ThermoFisher Scientific, Waltham, Mass., USA). The assay range for this ELISA is 3.13-200 pg/mL, and its analytical sensitivity 0.75 pg/mL. Detectable cross-reactivity with unrelated antibodies or other proteins has never been reported. Procedures were performed according to the manufacturer's instructions. ELISA samples were processed in duplicate with the average of results used as the data point for any given dose.

Statistical Analysis. Data analysis was performed using SPC for Excel (BPI Consulting, Katy, Tex., USA). The quantitative data are reported as mean and standard deviation (x±s.d.). The t-test was used for comparing differences between the two groups, with a p value <0.05 considered the cut off for statistical significance.

Results

Table 2 summarizes the effects on TNF-α of consuming the two CBD. Differences between them were quantitatively substantial and statistically highly significant (p<0.001) and non-overlapping. The outcome achieved with CBD in invention at 5 mg/kg CBD administration showed TNF-α levels lowered by approximately 70% with a single dose. In contrast, 5 mg/kg CBD in an edible oil did not differ from the control, and even a 50 mg/kg dose of CBD in an edible oil resulted in no relevant lessening of TNF-α levels.

TABLE 2

| | Mean TNF-α | 95% |
|---|---|---|
| Treatment | pg/mL Serum | Confidence+/− |
| Control - No CBD | 428.4 | 60.5 |
| CBD in an Edible Oil | | |
| 5 mg/kg | 429.3 | 66.0 |
| 25 mg/kg | 408.7 | 43.4 |
| 50 mg/kg | 389.8 | 77.8 |
| CBD in the Present Invention | | |
| 5 mg/kg | 104.9 | 55.4 |
| 25/mg kg | 110.3 | 45.2 |

Table 2. Impact of a CBD Formulations on TNF-α Levels in Serum. Single doses of either CBD in invention or CBD in an edible oil as well as a PBS control were administered, as were differing amounts of CBD. The resulting CBD levels substantially differed between CBD in invention and CBD in an edible oil, with differences between CBD in the present formulations and methods and CBD in an edible oil non-overlapping and highly significant (p<0.001).

Turning to FIG. 3, there is graphically portrayed the results for different dosing regimens of CBD in an edible oil and CBD in accordance with the present formulations and methods. Differences between TNF-α ELISA values from subjects that received CBD in the present formulations and methods and their counterparts with CBD in an edible oil were considerable and statistically significant in all cases (p<0.001). Equivalency between the two groups was essentially non-existent at any dose, and a bottom line was that TNF-α ELISA values were substantially reduced with CBD the present formulations and methods and very little, if at all, with CBD in an edible oil.

Particularly striking was the 70% reduction in TNF-α levels achieved with 5 mg/kg CBD in the present formulations and methods. This is contrasted by no observable attenuation of TNF-α with 5 mg/kg CBD in an edible oil. There was no difference in ELISA values, either arithmetically or statistically, between 5 mg/kg CBD in an edible oil and the control group. Furthermore, there were no statistically significant advantages to TNF-α reduction with higher doses of CBD in an edible oil. There were small, albeit non-significant, quantitative decreases in TNF-α levels that accompanied increased doses of CBD in an edible oil, and these minor differences could prove statistically significant with greater samples sizes. In short, the present formulations and methods render CBD into a medicament capable of substantial TNF-α reductions, whereas CBD in an oil, the way in which most CBD-containing preparations are administered, seems to offer limited or no benefit.

### Example Embodiments

In one example there is provided a method of modulating cytokine levels in a subject comprising: orally administering to the subject, at least one cannabinoid in a carrier and in an amount that reduces cytokine levels in the subject to a greater degree than an equivalent amount of the cannabinoid administered in an edible oil, or a cannabinoid administered from a carrier consisting essentially of, or containing only, an edible oil, such as olive oil.

In one example of such a method, the cytokine levels are reduced by more than 30% compared to a reduction provided by the cannabinoid administered in the edible oil.

In one example of such a method, the cytokine levels are reduced by between about 30% and 90% as compared to a reduction provided by the cannabinoid administered in the edible oil.

In one example of such a method, the cytokine levels are reduced by at least 70% as compared to a reduction provided by the cannabinoid administered in the edible oil.

In one example of such a method, the cytokine is a pro-inflammatory cytokine.

In one example of such a method, the pro-inflammatory cytokine is a member selected from the group consisting of: chemokines (CC), interferons (IFN), interleukins (IL), lymphokines (LK), tumor necrosis factors (TNF) or a combination thereof.

In one example of such a method, the pro-inflammatory cytokine is a tumor necrosis factor.

In one example of such a method, the tumor necrosis factor is TNF-α.

In one example of such a method, the pro-inflammatory cytokine is an interleukin.

In one example of such a method, the interleukin is IL-6.

In one example of such a method, the cannabinoid comprises cannabidiol (CBD).

In one example of such a method, the cannabinoid is administered in an amount of from about 5 mg/kg of the subject's body weight to about 50 mg/kg of the subject's body weight.

In one example of such a method, the carrier comprises a mixture of at least one oil and a combination of surfactants.

In one example of such a method, the mixture of surfactants includes at least one hydrophilic surfactant.

In one example of such a method, the amount of CBD in the carrier and the amount of CBD in the edible oil are the substantially the same.

In one example of such a method, the amount of CBD in the carrier is less than the amount of CBD in the edible oil.

In one example of such a method, the amount of CBD in the carrier is at least 3 times less to 10 times less than the amount of CBD in the edible oil.

In one example of such a method, the cytokine levels are manifest as a result of a cytokine dysregulation event.

In one example of such a method, the dysregulation event occurs in connect with at least one of: an autoimmune condition; a virally induced condition; a bacterially induced condition; or a wound.

In one example of such a method, the virally induced condition is a SARS-CoV-2 infection.

It is understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, materials, shape, form, function and manner of operation, assembly and use may be made without departing from the principles and concepts set forth herein.

What is claimed is:

1. A method of modulating cytokine levels in a subject comprising:
orally administering to the subject, at least one cannabinoid in a carrier and in an amount that reduces cytokine levels in the subject to a greater degree than an equivalent amount of the cannabinoid administered in an edible oil.

2. The method of claim 1, wherein the cytokine levels are reduced by more than 30% compared to a reduction provided by the cannabinoid administered in an edible oil.

3. The method of claim 1, wherein the cytokine levels are reduced by between about 30% and 90% as compared to a reduction provided by the cannabinoid administered in an edible oil.

4. The method of claim 3, wherein the cytokine levels are reduced by at least 70% as compared to a reduction provided by the cannabinoid administered in an edible oil.

5. The method of claim 1, wherein the cytokine is a pro-inflammatory cytokine.

6. The method of claim 5, wherein the pro-inflammatory cytokine is a member selected from the group consisting of: chemokines (CC), interferons (IFN), interleukins (IL), lymphokines (LK), tumor necrosis factors (TNF) or a combination thereof.

7. The method of claim 6, wherein the pro-inflammatory cytokine is a tumor necrosis factor.

8. The method of claim 7, wherein the tumor necrosis factor is TNF-α.

9. The method of claim 6, wherein the pro-inflammatory cytokine is an interleukin.

10. The method of claim 9, wherein the interleukin is Interleukin-6.

11. The method of claim 1, wherein the cannabinoid comprises cannabidiol (CBD).

12. The method of claim 1, wherein the cannabinoid is administered in an amount of up to about 50 mg/kg of the subject's body weight.

13. The method of claim 1, wherein the carrier comprises a mixture of at least one oil and a combination of surfactants.

14. The method of claim 1, wherein the mixture of surfactants includes at least one hydrophilic surfactant.

15. The method of claim 11, wherein the amount of CBD in the carrier and the amount of CBD in an edible oil are the substantially the same.

16. The method of claim 11, wherein the amount of CBD in the carrier is less than the amount of CBD in an edible oil.

17. The method of claim 11, wherein the amount of CBD in the carrier is at least 3 times less to 10 times less than the amount of CBD in an edible oil.

18. The method of claim 1, wherein the cytokine levels are manifest as a result of a cytokine dysregulation event.

19. The method of claim 18, wherein the dysregulation event occurs in connect with at least one of: an autoimmune condition; a virally induced condition; a bacterially induced condition; or a wound.

20. The method of claim 18, wherein the virally induced condition is a SARS-CoV-2 infection.
